(19) United States
(12) Patent Application Publication
(10) Pub. No.: US 2014/0214067 A1
(43) Pub. Date: Jul. 31, 2014

(54) PERCUTANEOUS TRANSLUMINAL ANGIOPLASTY DEVICE WITH INTEGRAL EMBOLIC FILTER

(71) Applicant: Contego Medical, LLC, Raleigh, NC (US)

(72) Inventors: Ravish Sachar, Raleigh, NC (US); Udayan G. Patel, San Jose, CA (US)

(73) Assignee: Contego Medical, LLC, Raleigh, NC (US)

(21) Appl. No.: 14/091,903

(22) Filed: Nov. 27, 2013

Related U.S. Application Data
(60) Provisional application No. 61/730,213, filed on Nov. 27, 2012.
(60) Provisional application No. 61/794,877, filed on Mar. 15, 2013.

(51) Int. Cl. A61F 2/01; A61M 25/10

(52) U.S. Cl. CPC A61F 2/013; A61M 25/104
USPC 606/194; 606/200

(57) ABSTRACT

A percutaneous transluminal angioplasty device includes an embolic filter mounted to the catheter shaft at a location distal to the angioplasty balloon. Thus the filter can be down-stream from the blockage and can be properly positioned to capture embolic particles that may be set loose into the blood stream as the angioplasty procedure can be performed. The embolic filter can be normally un-deployed against the catheter shaft to facilitate introduction and withdrawal of the device to and from the operative site. Once the angioplasty balloon can be properly positioned, however, means operatively associated with the embolic filter can be actuated to erect the filter to position a filter mesh across the lumen of the vessel.

### CROSS-REFERENCE TO RELATED APPLICATION

This application claims priority to U.S. provisional application Ser. No. 61/730,213 filed on Nov. 27, 2012. Additionally, this application is a continuation-in-part of U.S. patent application Ser. No. 11/763,118, filed Jun. 14, 2007, currently pending, which is a continuation-in-part of U.S. patent application Ser. No. 10/997,803, filed Nov. 24, 2004, now U.S. Pat. No. 8,403,976, which claims priority to Provisional Patent Application No. 60/813,395, filed Jun. 14, 2006. This application also claims priority to U.S. patent application Ser. No. 12/604, 236, filed on Oct. 22, 2009, currently pending, which claims priority to U.S. provisional application Ser. No. 61/107,391 filed on Oct. 22, 2008, U.S. provisional application Ser. No. 61/107,395 filed on Oct. 22, 2008 and U.S. provisional application Ser. No. 61/107,404 filed on Oct. 22, 2008.

### BACKGROUND

1. Field of the Invention

Implementations described herein relate generally to surgical devices and relate more specifically to percutaneous transluminal angioplasty devices.

2. Related Art

The vascular bed supplies a constant flow of oxygen-rich blood to the organs. In diseased vessels, blockages can develop that can reduce blood flow to the organs and cause adverse clinical symptoms up to and including fatality. Diseased vessels can comprise a range of material from early-stage thrombosis to late-stage calcified plaque.

Angioplasty can be described as a catheter-based procedure performed by a physician to open up a blocked vessel and restore blood flow. An entry site can be opened, for example, in the patient's groin, arm, or hand, and a guide wire and catheter can be advanced under fluoroscopic guidance to the location of the blockage. A catheter having a small balloon adjacent its distal end can be advanced under fluoroscopic guidance until the balloon lies within the stenosed region. The balloon can be then inflated and deflated one or more times to expand the stenosed region of the artery.

Angioplasty can release embolic particles down-stream from the stenosed location. These embolic particles can result in adverse clinical consequences. It has been shown beneficial to trap these embolic particles to prevent them from traveling downstream with blood flow to the capillary bed (e.g., Baim D S, Wahr D, George B, et al., Randomized trial of a distal embolic protection device during percutaneous intervention of saphenous vein aorta-coronary bypass grafts, Circulation 2002; 105:1285-90).

In addition to balloon angioplasty, stenoses can also be treated with stents and with mechanical thrombectomy devices. These devices can be also prone to releasing embolic particles downstream from the stenosed location.

Systems available today used to catch these embolic particles consist primarily of filter systems or occlusion balloon systems, both built on a guidewire. These systems suffer shortcomings related to simplicity of use and crossing tight lesions with a filter or balloon guidewire that can be larger in diameter than the guidewire which would normally be used. These embolic protection guidewires also suffer from flexibility and stability problems that render the protected angioplasty procedure relatively more difficult in many cases. In the case of saphenous vein grafts, the problems relate specifically to aorto-ostial lesions, where the guidewire may not be long enough to provide support, or distal vein graft lesions, where there can be not enough of a landing zone for the filter. The latter can be a problem as currently available filter systems can have a considerable distance between the treatment balloon and the distal filter. This distance can be a problem not only in distal vein graft lesions, but also in arterial stenoses in which there can be a side branch immediately after the stenosis. In such cases, the filter can often be deployed only distal to the side branch, thus leaving the side branch unprotected from embolic particles.

Accordingly, a need exists for improved percutaneous transluminal angioplasty devices having an integral embolic filter.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Stated generally, the present disclosure comprises a percutaneous transluminal angioplasty device with integral embolic filter. Because the filter can be integral with the catheter of the angioplasty device, any need to insert a separate device into the vessel can be eliminated. Further, proper placement of the angioplasty balloon can assure proper placement of the embolic filter.

Stated somewhat more specifically, the percutaneous transluminal angioplasty device of the present disclosure comprises an embolic filter mounted to the catheter shaft at a location distal to the angioplasty balloon, stent, mechanical thrombectomy device or the like. Thus, the filter can be positioned downstream from the blockage in order to capture embolic particles that may be set loose into the blood stream during the angioplasty procedure. The embolic filter can be un-deployed against the catheter shaft in an un-deployed position to facilitate introduction and withdrawal of the device to and from the operative site. Once the angioplasty balloon, stent, mechanical thrombectomy or like device is properly positioned, means operatively associated with the embolic filter can be actuated to erect the filter to position a filter mesh across the lumen of the coronary artery.

In some aspects, the means for deploying the filter can comprise a balloon which longitudinally displaces one end of the filter toward the other, causing longitudinal ribs to bow outward, thus deploying the filter mesh. In other aspects the means for deploying the filter comprises a balloon interposed within the proximal and distal ends of the filter, whereby inflating the balloon will bias the ribs away from the catheter shaft, causing the ribs to bow outwardly to erect the filter mesh. In still other aspects the means for deploying the filter comprises a pull wire attached to one end of the filter, such that pulling on the wire longitudinally displaces one end of the filter toward the other, causing longitudinal ribs to bow outward, thus deploying the filter mesh.

In yet other aspects of the invention, a reservoir can be provided at the distal tip of the filter so that when the device collapses for withdrawal, debris does not get pushed out of the filter.

Additional features and advantages of exemplary implementations of the disclosure will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates a partial cut away side view of a percutaneous transluminal angioplasty device according to a first aspect of the disclosed invention, with the angioplasty balloon and embolism filter in their un-deployed positions.

FIG. 2 illustrates a partial cut away side view of the percutaneous transluminal angioplasty device of FIG. 1 showing the angioplasty balloon and embolism filter in their deployed positions.

FIG. 3 illustrates a cross sectional view taken along line 3-3 of FIG. 1.

FIG. 4 illustrates a cross sectional view taken along line 4-4 of FIG. 1.

FIG. 5 illustrates a cross sectional view taken along line 5-5 of FIG. 1.

FIG. 6 illustrates a second aspect of a percutaneous transluminal angioplasty device according to the present invention, which differs from the percutaneous transluminal angioplasty of FIGS. 1 and 2 in that the actuation balloon can be on the proximal side of the embolic filter, and the filter deploys from a different direction.

FIG. 7 illustrates a view of the percutaneous transluminal angioplasty device of FIG. 6 showing the angioplasty balloon inflated and the embolic filter deployed.

FIG. 8 illustrates a third aspect of a percutaneous transluminal angioplasty device and differs from the previously described aspects in that the means for deploying the embolic filter can be a bellows. FIG. 8 shows the angioplasty balloon and the embolic filter in their un-deployed positions.

FIG. 9 illustrates another view of the percutaneous transluminal angioplasty device of FIG. 8 showing the angioplasty balloon and the embolic filter in their deployed positions.

FIG. 10 illustrates another aspect of a percutaneous transluminal angioplasty device according to the present disclosure which employs a bellows to raise and lower the embolic filter. The aspect of FIG. 10 differs from the aspect of FIGS. 8 and 9 in that the bellows can be disposed on the distal end of the filter such that the filter deploys from the opposite direction. FIG. 10 shows the angioplasty balloon and the embolic filter in their un-deployed positions.

FIG. 11 illustrates another view of the percutaneous transluminal angioplasty device of FIG. 10, showing the angioplasty balloon and the embolic filter deployed.

FIG. 12 illustrates still another aspect of a percutaneous transluminal angioplasty device according to the present invention, in which the balloon interposed between the catheter shaft and the ribs forces the ribs upward, thereby causing the embolic filter to deploy. FIG. 12 shows the device with the angioplasty balloon and the embolic filter in their un-deployed configurations.

FIG. 13 illustrates another view of the percutaneous transluminal angioplasty device of FIG. 12, showing the angioplasty balloon and the embolic filter in their deployed configurations.

FIG. 14 illustrates another aspect of a percutaneous transluminal angioplasty device according to the present invention. This aspect differs from the aspects of FIGS. 12 and 13 in that the balloon can be located at the opposite end of the filter. Nonetheless, when inflated, the balloon forces the ribs away from the shaft and into their arcuate positions, thereby deploying the embolic filter. FIG. 14 shows the aspect with the angioplasty balloon un-deployed and the embolic filter retracted against the catheter shaft.

FIG. 15 illustrates another view of the aspect of FIG. 14, showing the angioplasty balloon inflated and the embolic filter deployed.

FIG. 16 illustrates still another aspect of a percutaneous transluminal angioplasty device according to the present invention. This aspect employs a pull wire operable from outside the patient which can be attached to a distal ring of the embolic filter. When the physician exerts tension on the wire, the distal ring can be displaced proximally, bringing it closer to the proximal ring, causing the ribs to bow outward and thereby deploying the embolic mesh filter. FIG. 16 shows the device with the angioplasty balloon deflated and the embolic filter un-deployed against the catheter shaft.

FIG. 17 illustrates a different view of the aspect of FIG. 16 and shows the angioplasty balloon inflated and the embolic filter deployed.

FIG. 18 illustrates another aspect of a percutaneous transluminal angioplasty device according to the present invention, showing the angioplasty balloon and the embolic filter in their un-deployed conditions. Optionally, the disclosed embolic filter can be formed from a shape memory material in which the base or unstrained shape memory is in a baseline open position or a baseline closed position.

FIG. 19 illustrates another view of the aspect of FIG. 18, showing the angioplasty balloon inflated and the embolic filter raised to an open position.

FIG. 20 illustrates yet another aspect of a percutaneous transluminal angioplasty device according to the present invention, showing the angioplasty balloon and the embolic filter in their un-deployed configurations.

FIG. 21 illustrates another view of the aspect of FIG. 20, showing the angioplasty balloon inflated and the embolic filter deployed.

FIG. 22 illustrates a side cut away view of a coronary artery with a stenosis.

FIG. 23 illustrates the coronary artery of FIG. 20 with a guide wire fed through the coronary artery and through the stenosis.

FIG. 24 illustrates the device of FIG. 1 threaded over the guide wire of FIG. 23 and positioned such that the angioplasty balloon can be located within the stenosis.

FIG. 25 illustrates the angioplasty balloon in its deployed configuration to reduce the stenosis, and the embolic filter deployed to capture any embolic particles that may break loose into the blood stream as a result of the angioplasty procedure.

FIG. 26 illustrates a partial cut away side view of an aspect of a device in which the angioplasty balloon and embolism filter, shown in their un-deployed positions, can be reversed on the catheter shaft for peripheral vascular applications in which blood flows in the opposite direction.

FIG. 27 illustrates a partial cut away side view of the device of FIG. 26 showing the angioplasty balloon and embolic filter in their deployed positions.

FIG. 28 illustrates a side view of an embolism filter according to another aspect of the present invention.

FIG. 29 illustrates a side view of the embolism filter of FIG. 28 with the inflation balloon and the embolic filter in their deployed configurations. The filter mesh is shown removed to reveal interior detail.

FIG. 30 illustrates a side view of the embolic filter of FIG. 28 with the inflation balloon deflated. The filter mesh is shown removed to reveal interior detail.

FIG. 31 illustrates a side view of the embolic filter of FIG. 28 being retracted into the forward end of a catheter to collapse the filter. The filter mesh is shown removed to reveal interior detail.

FIG. 32 illustrates a side view of the embolic filter of FIG. 28 with the filter deployed and the filter mesh shown.

FIG. 33 illustrates a side cutaway view of another aspect of an angioplasty device showing an angioplasty balloon and an embolic filter in their un-deployed configurations.

FIG. 34 illustrates a side cutaway view of the angioplasty device of FIG. 33 showing the angioplasty balloon and the embolic filter deployed.

FIG. 35 illustrates a side view of a further aspect of an angioplasty device in which the filter mesh extends beyond the end of the ribs so as to form a sac when the frame is in an un-deployed configuration.

FIG. 36 illustrates a side view of the angioplasty device of FIG. 35 when the frame is in an un-deployed configuration.

FIG. 37 illustrates a generally cylindrical filter frame shown unrolled and flattened.

FIG. 38 illustrates the generally cylindrical filter frame of FIG. 37 shown in a deployed configuration without the filter membrane.

FIG. 39 illustrates the generally cylindrical filter frame of FIG. 37 shown in a deployed configuration with the filter membrane.

FIG. 40 illustrates an alternate frame design that is generally cylindrical shown unrolled and flattened.

FIG. 41 illustrates another alternate frame design that is generally cylindrical shown unrolled and flattened.

FIG. 42 illustrates a top view an aspect of a filter membrane formed in the shape of a funnel.

FIG. 43 illustrates a side view of an alternate frame design that is generally cylindrical shown unrolled and flattened.

FIG. 44 illustrates a perspective view of an alternate frame design shown unrolled and flattened that is generally cylindrical when formed.

FIG. 45 illustrates the alternate frame design of FIG. 45 shown stretched out over a mandrel to form a substantially cylindrical shape.

FIG. 46 illustrates the alternate frame design of FIG. 45 showing the respective ends of the frame being trimmed to form a desired elongate longitudinal length. The trimmed cylindrically shaped frame design can be heat treated to set the shape memory in the formed position or, optionally, the trimmed frame can be compressed to a desired dimension and then heat treated to set the shape memory in the formed position (baseline closed position) or it can be expanded to a desired dimension and then heat treated to set the shape memory in the formed position (baseline open position).

FIG. 47 illustrates the alternate frame design of FIG. 45 showing the trimmed cylindrically shaped frame design heat treated to set the shape memory in the baseline closed position upon application of an axial compression, for example and not meant to be limiting, by an external operator pulling on a pull wire to controllably cause the mid-portion of the trimmed cylindrically shaped frame design to expand to a desired diameter, which is a multiple of the original diameter of the frame design in the baseline closed position.

### DETAILED DESCRIPTION OF THE INVENTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well-known aspects of percutaneous transluminal angioplasty devices and embolic filters have not been described in particular detail in order to avoid unnecessarily obscuring aspects of the disclosed implementations.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

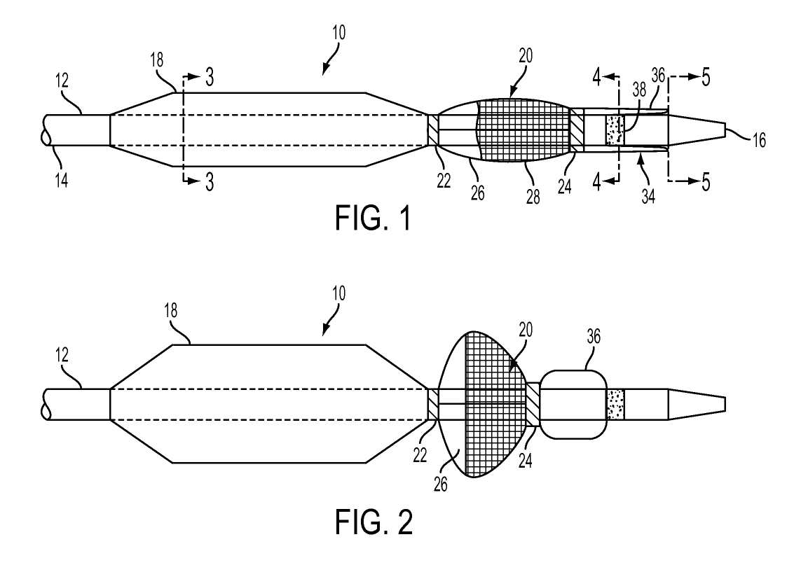

Referring now to the drawings, in which identical numbers indicate identical elements throughout the various views, FIGS. 1 and 2 illustrate a first aspect of a percutaneous transluminal angioplasty device 10 according to the present invention. The device 10 comprises an elongated catheter 12 having a shaft 14 with a proximal end (not shown) and a distal end 16. Spaced a short distance proximally from the distal end 16 of the catheter 12 can be an angioplasty balloon 18 of conventional design. In FIG. 1 the angioplasty balloon 18 can be shown in a deflated or un-deployed condition. In FIG. 2 the angioplasty balloon 18 can be shown in an inflated or deployed condition.

Located between the angioplasty balloon 18 and the distal tip 14 of the catheter 12 can be a collapsible filter 20. The filter 20 can include a proximal ring portion 22 and a distal ring portion 24. A plurality of elongated ribs 26 extend generally longitudinally between the proximal and distal rings 22, 24. These ribs can be made of a shape memory material, such as nitinol, and in their baseline position, these ribs can be un-deployed. A filter mesh 28 overlies the distal portion of the ribs 26. In the aspect of FIGS. 1 and 2, the distal ring 24 can be movable toward and away from the proximal ring 22. As the distal ring 24 moves toward the proximal ring 22, the ribs 26 bow outward. As the ribs 26 bow outward, the filter mesh 28 overlaying the ribs can be deployed. FIG. 1 shows the filter 20 in its un-deployed condition, while FIG. 2 shows the filter in its deployed condition.

Means 34 can be included for deploying and collapsing the filter 20 of the device 10 shown in FIGS. 1 and 2. Specifically a balloon 36 can have its distal end 38 bonded to the shaft 14 of the catheter 12. When the distal ring 24 is in its withdrawn position, as shown in FIG. 1, the bulk of the balloon 36 can be folded forward over the shaft 14 of the catheter 12. When the balloon 36 is deployed, as shown in FIG. 2, the balloon 36 can expand proximally, pushing the distal ring 24 in a proximal direction, causing the ribs 26 to bow outward and thereby deploying the filter 20. When the balloon 32 is deflated, the shape memory ribs can straighten, urging the distal ring 24 in a distal direction and collapsing the filter 20 to its un-deployed configuration close to the shaft 14 of the catheter 12.

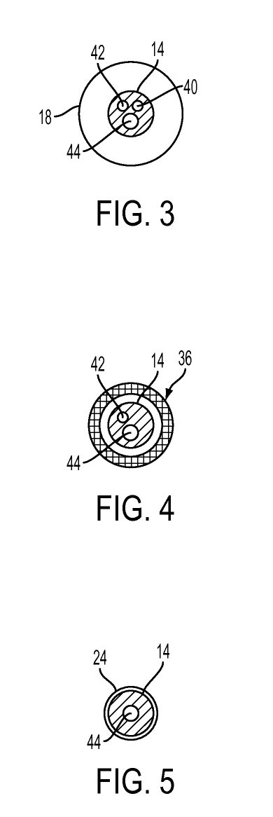

FIGS. 3, 4, and 5 show cross sections of the device 10 at various locations along its length. Referring first to FIG. 3, the catheter shaft 12 has three lumens: two smaller lumens and a large main lumen. The two smaller lumens can be inflation lumens, one lumen 40 for the angioplasty balloon 18, and one lumen 42 for the balloon 36 which controls the filter 20. The larger main lumen 44 can be used to receive a guide wire (not shown) over which the device 10 can be advanced to position the device for performing an angioplasty procedure.

Referring now to FIG. 4, this cross section illustrates a location distal to the angioplasty balloon 18. Consequently, the angioplasty balloon inflation lumen 40 has terminated and is no longer visible. Thus, FIG. 4 shows only two lumens, the main lumen 44 for receiving the guide wire, and the smaller inflation lumen 42 for the filter balloon 36.

Referring now to FIG. 5, this cross section illustrates a location distal to the filter balloon 36, and hence only the main lumen 44 can be visible.

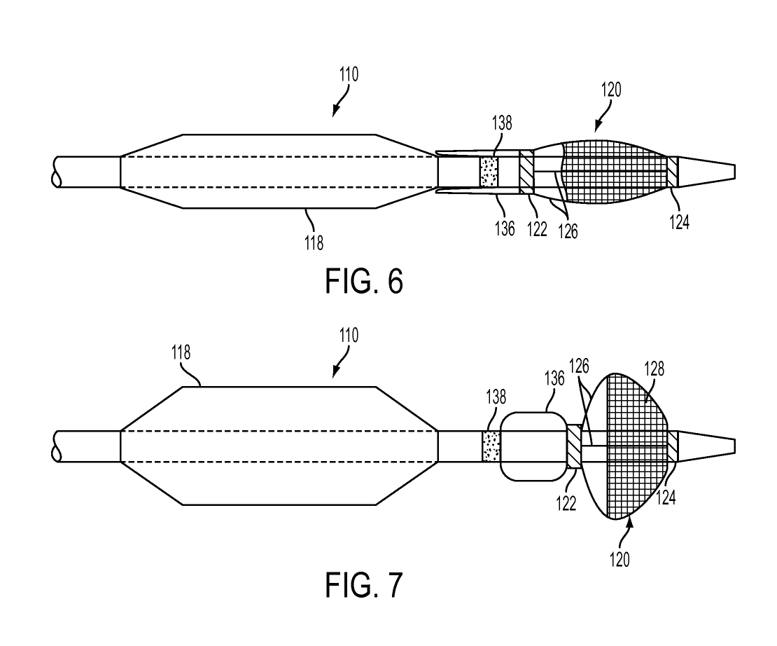

FIGS. 6 and 7 show an alternate aspect of a percutaneous transluminal angioplasty device 110 according to the present invention. This device can be similar to the device 10 previously described, with the exception that the filter 120, in this case, has its distal ring 124 fixed, and the proximal ring 122 of the filter 120 can be movable toward and away from the distal ring to cause the ribs 126 to bow outwardly or to straighten. The balloon 136 can be located on the proximal side of the filter 120 and pushes the proximal ring 122 in a distal direction when the balloon 136 can be inflated.

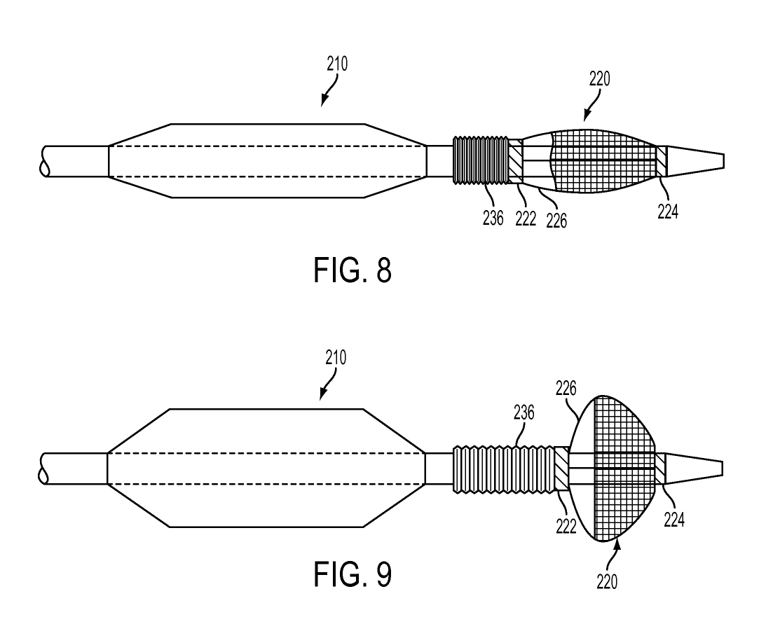

Referring now to FIGS. 8 and 9, yet another alternate aspect of a percutaneous transluminal angioplasty device 210 can be shown. This device can be similar to the device shown in FIGS. 1 and 2, with the exception that the means for deploying the filter 220 can be a bellows 236, instead of a balloon. In FIG. 8, the bellows 236 can be deflated and hence it can be in an un-deployed condition, permitting the ribs 226 of the filter 220 to straighten out against the shaft 214 of the catheter 212 in an un-deployed state. In FIG. 9, the bellows 236 has been inflated, pushing the proximal ring 222 in a distal direction, bowing out the ribs 236 and deploying the filter mesh 238.

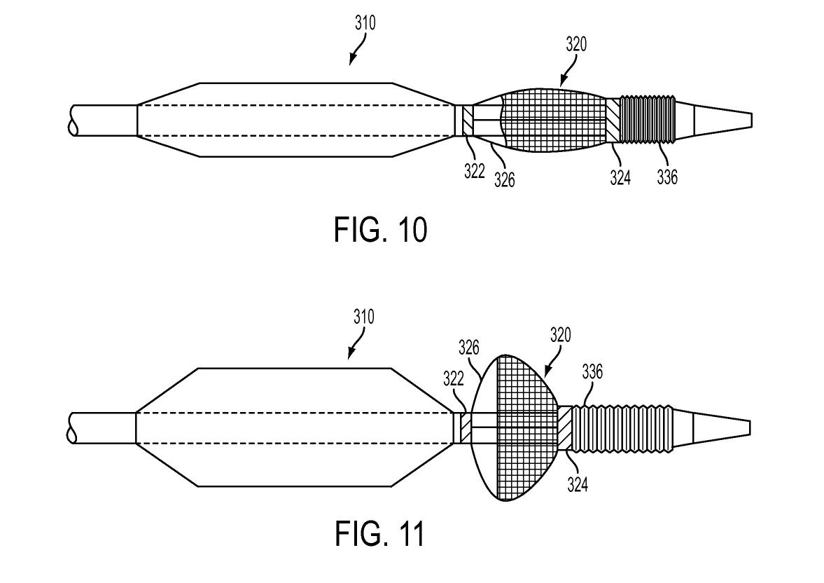

FIGS. 10 and 11 illustrate still another aspect of a percutaneous transluminal angioplasty device 310. This device can be similar to the device shown in FIGS. 8 and 9, with the exception that the bellows 336 can be placed on the distal side of the filter 320. Thus, when the bellows 336 can be inflated, it moves the distal ring 324 in a proximal direction toward the proximal ring 322, thereby causing the ribs 326 to bow outwardly, deploying the filter mesh 338.

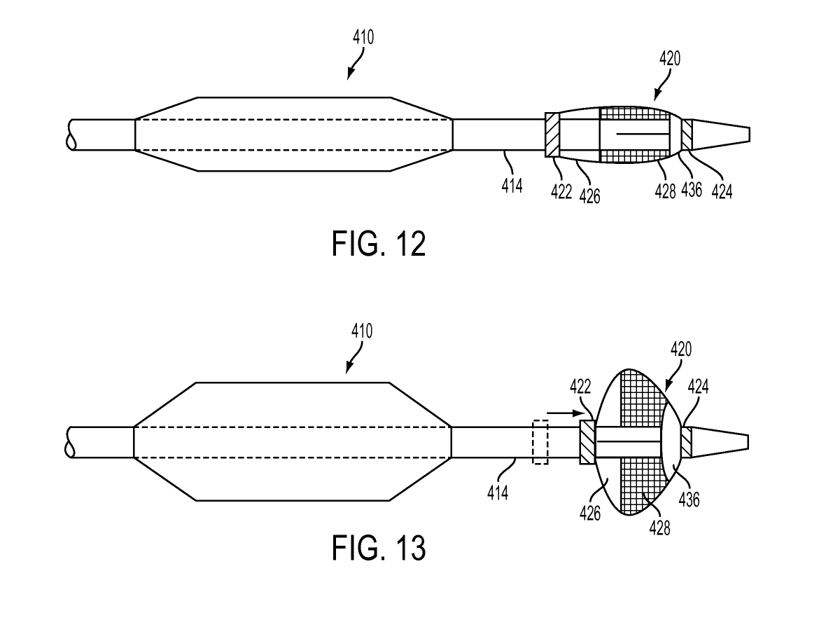

FIGS. 12 and 13 depict another aspect of a percutaneous transluminal angioplasty device 410. In this device the means for deploying the filter comprises a balloon 436 disposed between the catheter shaft 414 and the ribs 426 adjacent the fixed distal ring 424 of the filter 420. When the balloon 436 can be inflated, it forces the ribs 426 outward away from the catheter shaft 414, thereby bowing the ribs and drawing the proximal ring 422 of the filter 420 in a distal direction. As the ribs 426 bow outward, the filter mesh 428 can be deployed, thereby raising the filter 420.

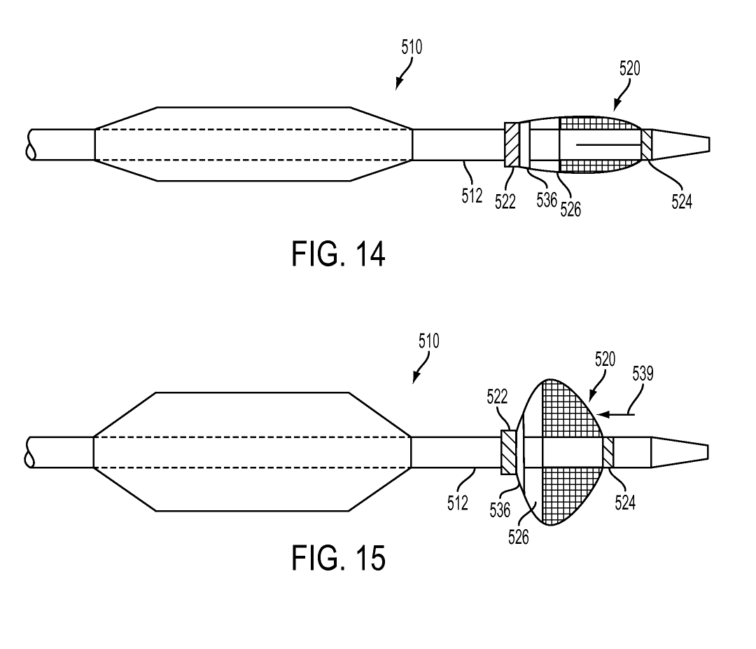

FIGS. 14 and 15 show a device 510 similar to that shown in FIGS. 12 and 13, with the exception that the balloon 536 can be placed between the catheter shaft 512 and the ribs 526 adjacent the proximal ring 522 of the filter 520. In the device 510, the distal ring 524 can be free to slide along the catheter shaft 512, such that when the balloon 536 can be inflated and forces the ribs 526 to bow outward, the distal ring 524 slides in a proximal direction, as indicated by the arrow 539 as shown in FIG. 15, causing the filter 520 to deploy.

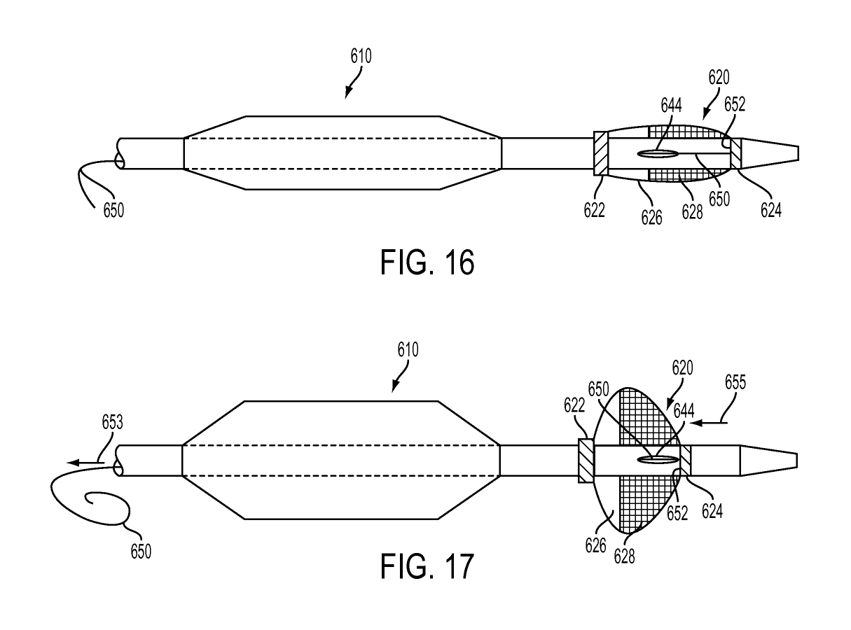

The aspect 610 shown in FIGS. 16 and 17 employs a different means for deploying the filter 620. In the aspect 610 a pull wire 650 can be used. The pull wire 650 can extend through what would formerly have been used as the filter balloon inflation lumen 644, and the distal end 652 of the pull wire 650 can be attached to the distal ring 624. When the physician wishes to deploy the filter 620, he exerts a tension on the wire 650, as indicated by the arrow 653, thus drawing the distal ring 624 in a proximal direction as indicated by the arrow 655 toward the proximal ring 622. The ribs bow 626 outward, deploying the filter mesh 628 as shown in FIG. 17.

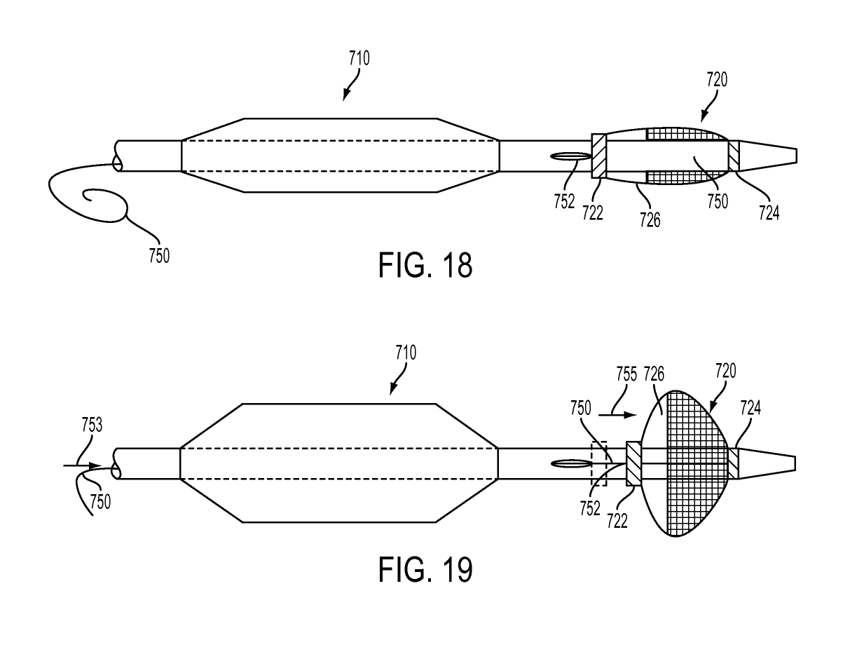

In the device 710 shown in FIGS. 18 and 19, the distal end 752 of a push wire 750 can be attached to the proximal ring 722. Thus when the wire 750 can be pushed in the direction indicated by the arrow 753, the proximal ring 722 can be advanced distally toward the distal ring 724 in the direction indicated by the arrow 755, causing the ribs 726 to bow outward and thereby deploying the filter 720, as shown in FIG. 19. Optionally, the disclosed embolic filter can be formed from a shape memory material in which the base or unstrained shape memory is in a baseline open position or a baseline closed position.

In one aspect, when the embolic filter has a baseline open configuration, the wire can be configured to attach to a portion of the proximal movable collar. In this aspect, it will be appreciated that the coupled wire will be held in tension when the catheter is inserted into the body to provide the desired degree of minimal cross-sectional area. Subsequently, when the embolic filter is positioned in the desired location within the patient's blood vessels, the tension can be selectively reduced or released, which will allow for the expansion or opening of the embolic filter as the shape memory material urges or biases the embolic filer to its baseline open position. It is contemplated that once the surgical procedure is complete, i.e., when an exemplary angioplasty procedure has been performed, the wire can be placed under tension and retracted, which pulls the proximal collar proximally and thereby closes the embolic filter.

In a similar aspect, where the embolic filter has a baseline closed position, it will be appreciated that the coupled wire will not need be held in tension when the catheter is inserted into the body to provide the desired degree of minimal cross-sectional area. When the embolic filter is positioned in the desired location within the patient's blood vessels, the wire is placed in compression to advance the activation wire is a forward or distal direction to effect the desired opening of the filter, which acts against the bias force that otherwise urges the embolic filter to the baseline closed position. In this aspect, it is contemplated that once the surgical procedure is complete, the compression force being externally applied to the wire can be released, which allows the shape memory of embolic filter to urge or bias the embolic filer to its baseline closed position and thereby close the embolic filter.

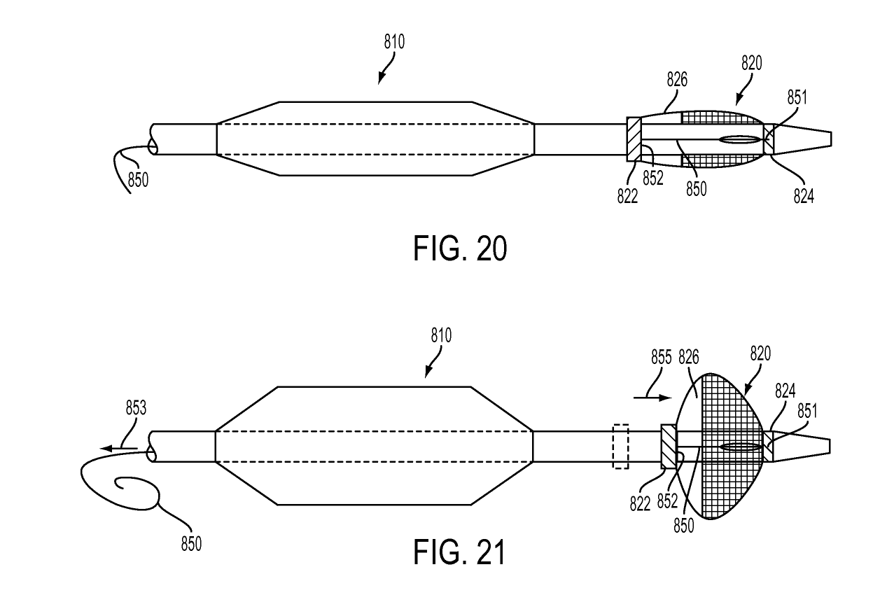

The device 810 shown in FIGS. 20 and 21 uses a pull wire 850 to erect the filter 820. The pull wire 850 can wrap around an opening 851 in the stationary distal ring 824 and can extend rearward toward the proximal ring 822 to which the distal end 852 of the pull wire can be attached. Thus when tension can be exerted on the pull wire 850 in the direction indicated by the arrow 853, the proximal ring 822 can be drawn distally toward the distal ring 824 in the direction indicated by the arrow 855, causing the ribs 826 to bow outward and thereby deploying the filter 820, as shown in FIG. 21.

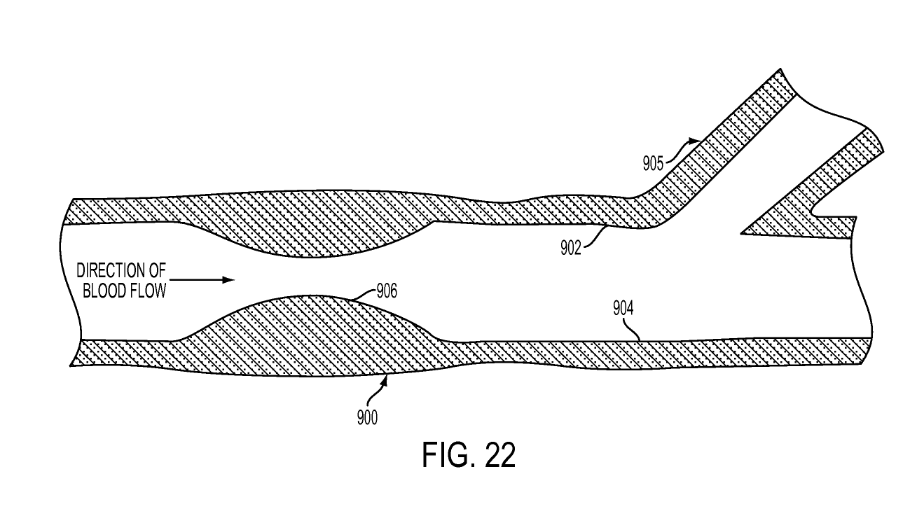

The operation of the device 10 will now be explained with respect to FIGS. 22-25, and it should be understood that the other devices operate on a substantially the same principles. FIG. 22 shows a vascular structure (e.g., coronary artery, saphenous vein graft, renal artery, carotid artery, superficial femoral artery, etc.) 900 with upper and lower walls 902, 904, a branch vessel 905, and a stenosis or blockage 906 caused by the build-up of plaque or other substances on the arterial walls in such a way as to narrow the diameter of the arterial lumen, and in the process, constrict the flow of blood therethrough.

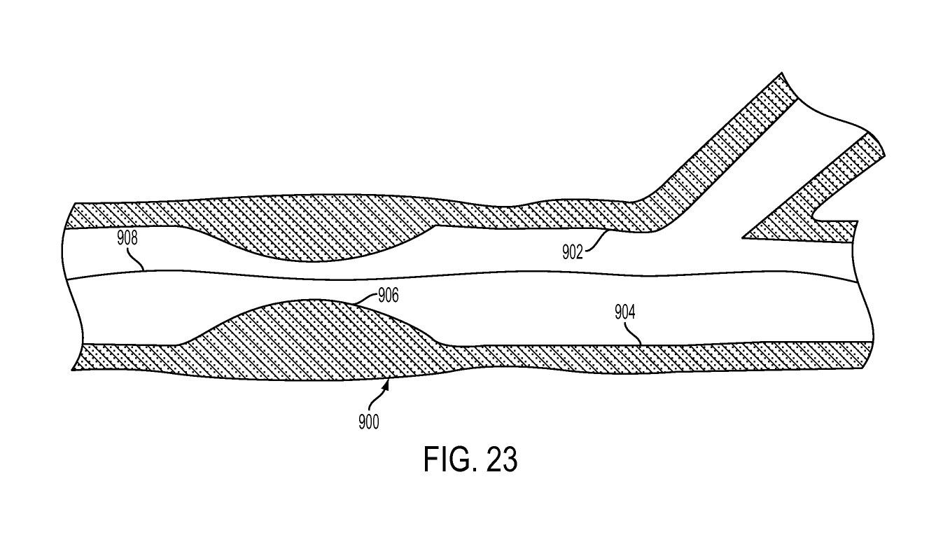

In FIG. 23, a guide wire 908 has been inserted by the physician, such as through the femoral artery, and guided through the vascular system until the guide wire passes through the stenosis 906 in the vascular structure 900.

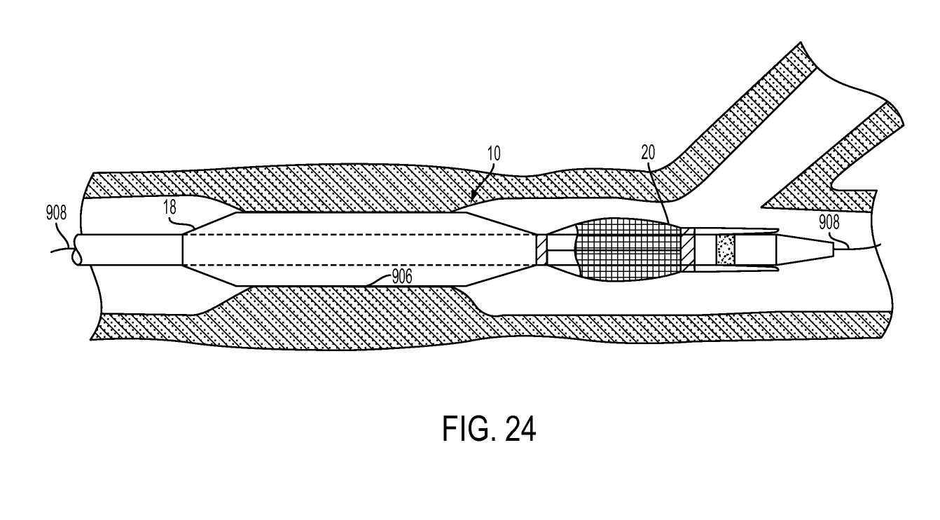

Referring now to FIG. 24, the apparatus 10 has been inserted over the guide wire 908 and advanced to a location wherein the angioplasty balloon resides within the stenosis 906. The embolic filter 20 resides a few centimeters distal or downstream from the angioplasty location. In FIG. 24 both the angioplasty balloon and the embolic filter can be shown in their un-deployed configurations.

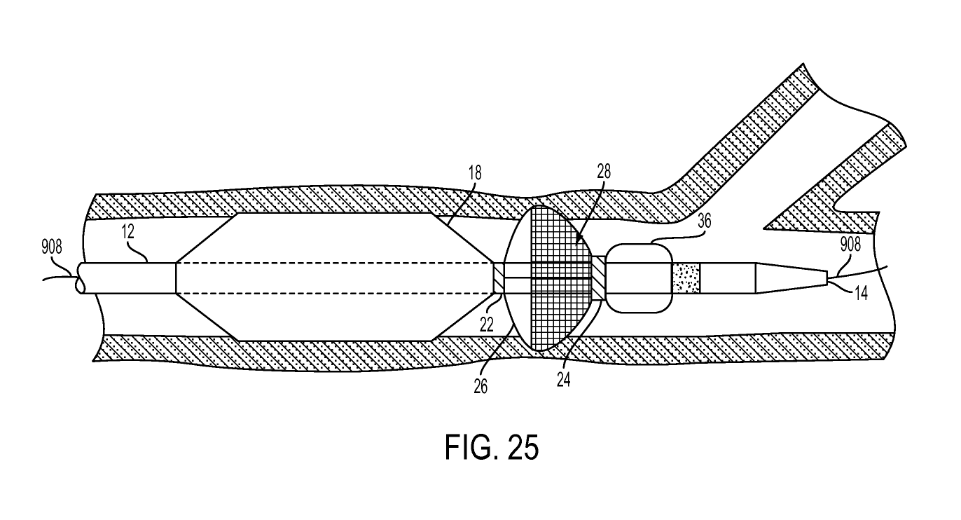

In FIG. 25 the embolic filter 20 has been deployed by inflating the filter balloon 36, causing the distal ring 22 to slide in a proximal direction along the catheter shaft 12. As the ribs 26 bow outward, the mesh filter material 28 supported by the ribs spreads so as to cover substantially the entire arterial lumen. The angioplasty balloon 18 can be deployed next. As the balloon 18 inflates, it pushes tissue and plaque forming the stenosis 906 outward, opening the stenosis and potentially loosening embolic particles in the process. Any such embolic particles which get released into the blood stream will be caught by the embolic filter 20 and will thereby be prevented from traveling to a location where they can cause injury to the patient.

FIG. 25 illustrates the close proximity in which the filter 20 can be deployed relative to the stenosis 906. Despite the short “landing area”, defined as the area between the stenosis 906 and the branch vessel 905, the filter 20 can be deployed to capture embolic particles upstream of the branch vessel.

When removing the device 10 from the coronary artery, the preferred procedure can be to deflate the angioplasty balloon 18 first, prior to collapsing the embolic filter 20. In this way, any embolic particles that break loose as the angioplasty balloon 18 deflates can be captured by the filter 20. The embolic filter balloon 20 can then be deflated, permitting the ribs 26 and filter mesh 28 to collapse against the shaft 14 of the catheter 12. Any embolic particles captured by the mesh 28 can be trapped against the shaft 14. The device 10 can be then withdrawn over the guide wire 908 and removed from the patient's body.

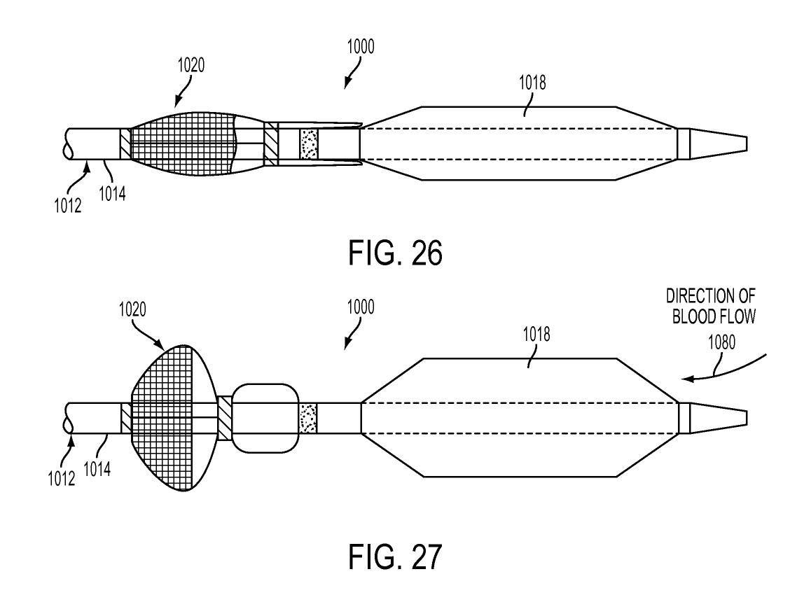

In various peripheral vascular applications, it may be necessary to insert the catheter against the direction of blood flow (e.g., the aorta). FIGS. 26 and 27 illustrate a device 1000 in which the angioplasty balloon 1018 and the embolic filter 1020 can be reversed on the shaft 1014 of the catheter 1012. Thus with the blood flowing within the vessel in the direction indicated by the arrow 1080, the embolic filter 1020 can be proximal to the angioplasty balloon 1018 and thus positioned to capture any embolic particles that may be dislodged by the angioplasty balloon.

While the aspect 1000 of FIGS. 26 and 27 employs the same method and device for deploying the embolic filter as the aspect 10 of FIGS. 1-3, it can be understood that the methods and devices for deploying the embolic filter of other aspects disclosed above can be equally applicable to a configuration like the device of aspect 1000 where the angioplasty balloon can be positioned between the embolic filter and the tip of the device.

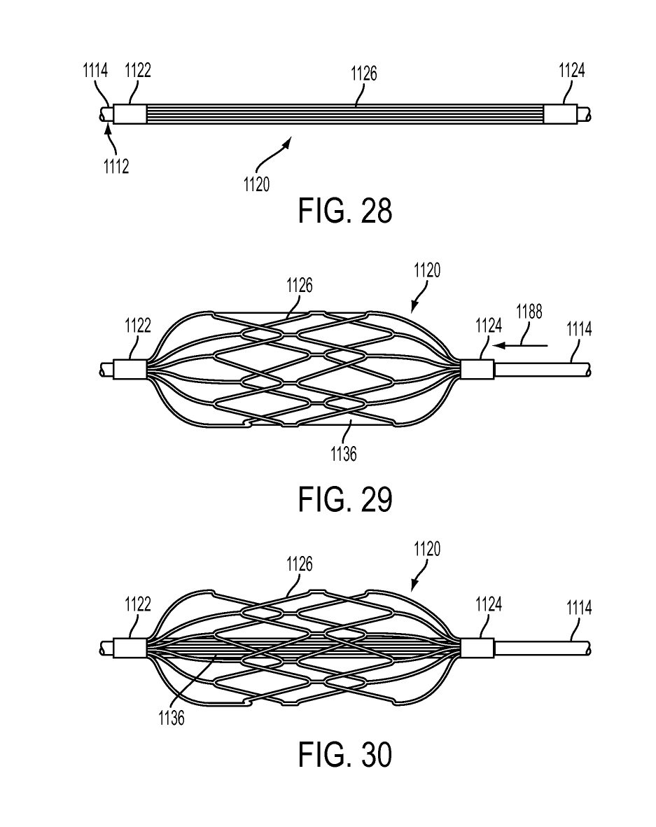

FIGS. 28-32 show still another aspect of an embolic filter 1120 for use in conjunction with an angioplasty balloon. FIGS. 28-32 show only the embolic filter 1120 and not the angioplasty balloon, but it can be understood that the embolic filter can be located on the same catheter 1114 as the angioplasty balloon in the same manner as the aspects previously disclosed. Further, FIGS. 29-31 show the embolic filter 1120 without its filter mesh 1128 for clarity of illustration.

In FIG. 28 the embolic filter 1120 can be folded closely against the shaft 1114 of the catheter 1112. The ribs 1126 of the filter 1120 extend between a proximal ring portion 1122 and a distal ring portion 1124. The distal ring portion 1124 can be slideably mounted on the shaft 1114 of the catheter 1112 and the proximal ring portion 1122 can be fixed with relation to the shaft of the catheter. In FIG. 29 the embolic filter balloon 1136 has been inflated, expanding the ribs 1126 of the embolic filter. As the ribs expand, the distal ring portion 1124 slides in the proximal direction, as shown by the arrow 1188. Once expanded, the ribs 1126 maintain their shape, such that when the embolic filter balloon 1136 deflates as shown in FIG. 30, the embolic filter 1120 remains expanded.

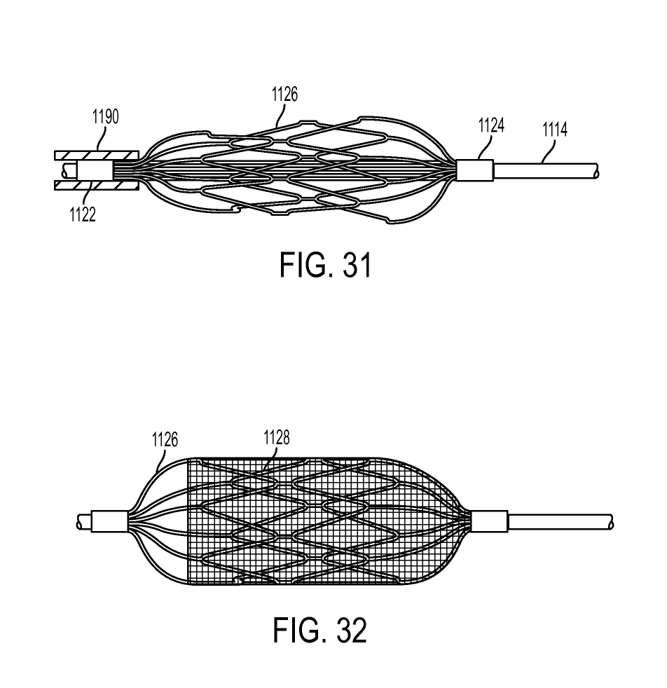

To retract the embolic filter 1120, a second, outer catheter 1190 can be advanced over the catheter 1112, as shown in FIG. 31, causing the ribs 1126 to collapse as the embolic filter can be withdrawn into the forward end of the outer catheter 1190. As the ribs 1126 collapse, the distal ring portion 1124 slides in the distal direction. Once the embolic filter 1120 has been completely retracted into the forward end of the outer catheter 1190, the outer and inner catheters can be withdrawn simultaneously.

FIG. 32 shows the embolic filter 1120 with filter mesh 1128 positioned over the ribs 1126.

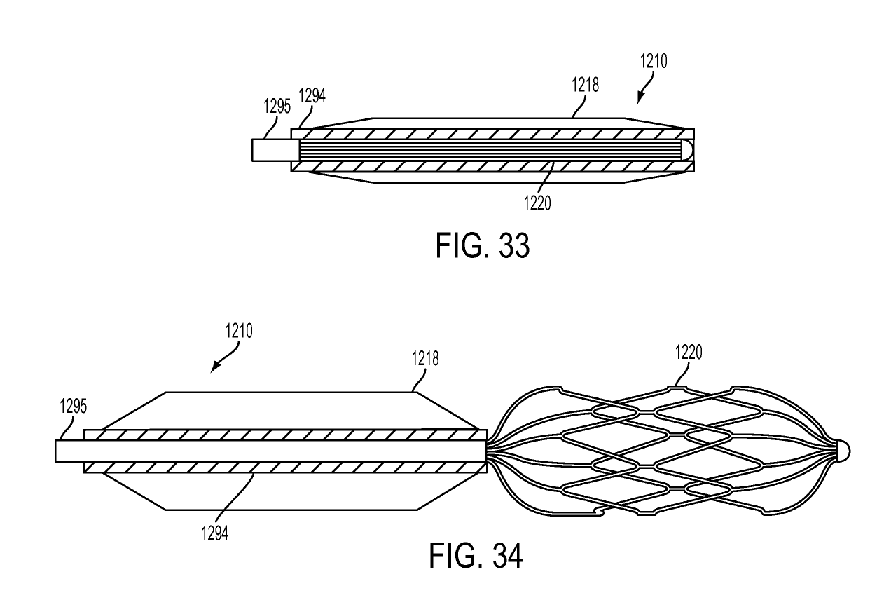

FIGS. 33 and 34 illustrate a further aspect of a percutaneous angioplasty device 1210, in which the embolic filter 1220 can be located on a different carrier than the angioplasty balloon 1218. Specifically, the angioplasty balloon 1218 can be located on an outer catheter 1294, and the embolic filter 1220 can be located at the forward end of an inner catheter 1295. (The embolic filter 1220 is shown without filter mesh in FIGS. 33 and 34 for clarity of illustration.) The outer catheter preferably has three lumens, one for inflating the angioplasty balloon 1218, one for accommodating a guide wire (not shown), and one for receiving the inner catheter 1295 and embolic filter 1220. The inner catheter 1295 can be slideably and telescopically disposed within the outer catheter 1294. The ribs 1226 of the embolic filter 1220 can be formed from a shape-memory metal such as nitinol and can be constructed to normally assume an “open” configuration. When retracted within the forward end of the outer catheter 1294, the ribs 1226 of the embolic filter collapse.

To use the percutaneous angioplasty device 1210, the inner catheter can be inserted into the outer catheter so that the embolic filter 1220 lies within the distal end of the device, as shown in FIG. 33. The outer and inner catheters 1294, 1295 can be inserted together, such as through the femoral artery, over a guidewire and advanced through the vascular system to a location wherein the deflated angioplasty balloon 1218 resides within the stenosis. Once location of the angioplasty balloon 1218 within the stenosis has been verified by suitable medical imaging technology, the inner catheter can be advanced to move the embolic filter 1220 beyond the forward end of the outer catheter 1294. As the embolic filter 1220 exits the confines of the outer catheter 1294, the ribs can assume their expanded configuration and deploy the embolic filter. Thereafter the angioplasty balloon 1218 may be deployed to treat the stenosis, and any emboli loosened during the procedure can be captured by the embolic filter 1220 downstream of the stenosis.

When the angioplasty procedure has been completed, the angioplasty balloon 1218 can be deflated, and the embolic filter 1220 can be withdrawn back into the forward end of the outer catheter 1294, collapsing the filter. The outer and inner catheters 1294, 1295 can be then withdrawn together from the patient.

In the foregoing aspect a wire can be substituted for the inner catheter 1295 as a means for carrying the embolic filter 1220.

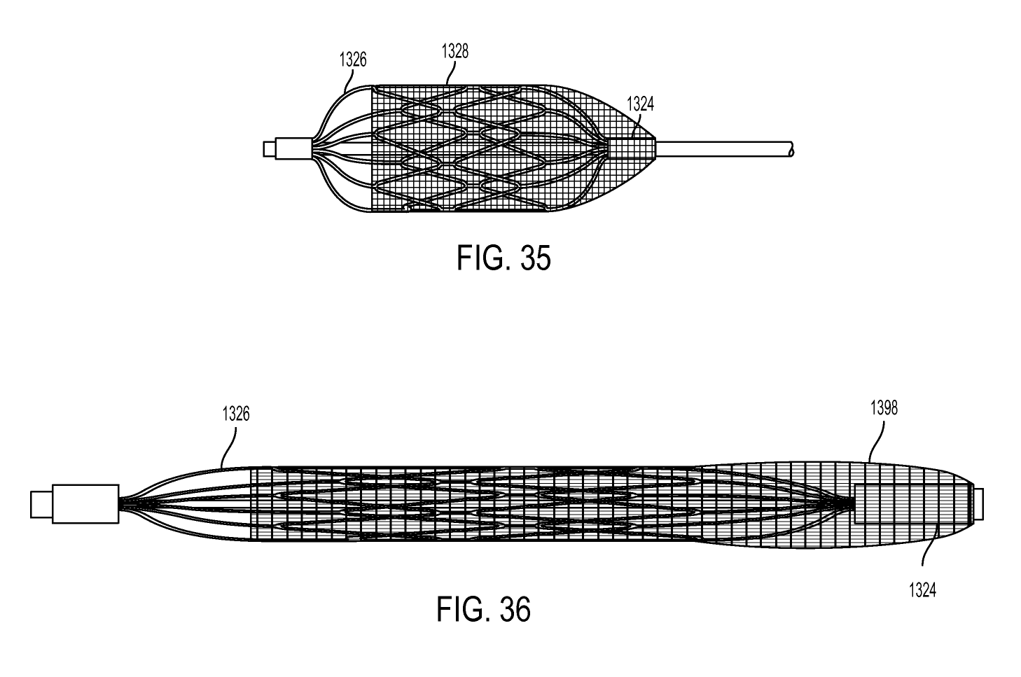

FIGS. 35 and 36 show an angioplasty device 1310 that can be identical to the device 10, with the exception that the filter mesh 1328 extends distally beyond the end of the ribs 1326 and can be attached to the distal end of the distal ring 1324. When the filter 1320 is un-deployed, as shown in FIG. 36, a sac 1398 can be formed which helps contain the embolic particles, thereby minimizing the possibility that the ribs 1326 will squeeze the particles out of the filter.

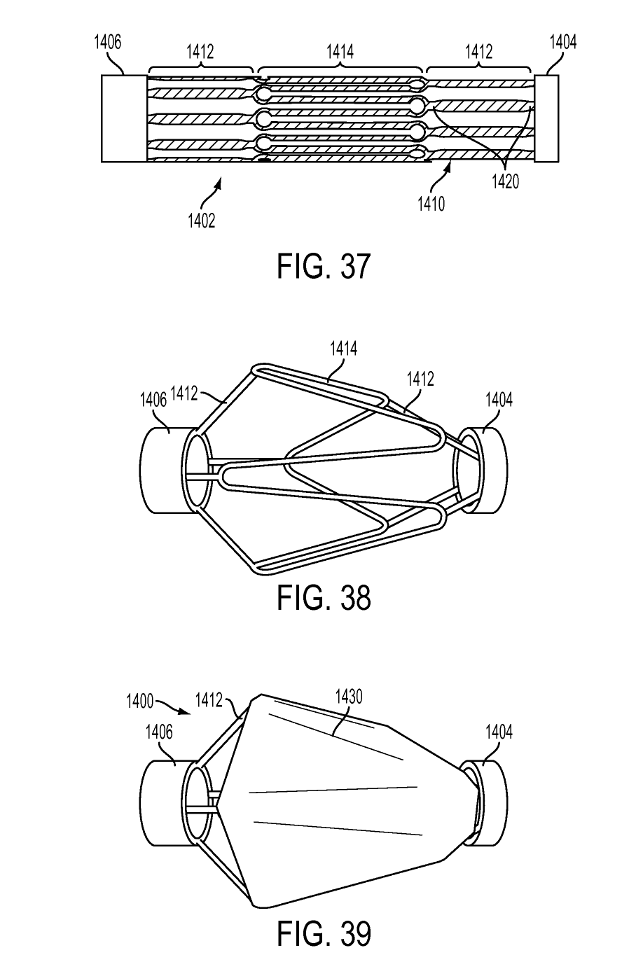

Referring now to FIGS. 37-39, an alternate aspect of a filter 1400 is illustrated. FIG. 37 can be a projection of a cylinder, i.e., a generally cylindrical filter frame 1402 can be shown unrolled and flattened. The frame 1402 can be made of flexible material such as Nitinol. The support frame 1402 can comprise a generally tubular shape with a proximal ring 1404 at one end and a distal ring 1604 at the opposite end. Struts 1410 can be attached between the end rings 1404, 1406. In some of the figures the struts are shown in solid black to facilitate differentiation between the struts and the spaces there between.

More specifically, the struts 1410 of the frame 1402 comprise a plurality of longitudinal struts 1412 at each end of the frame and a connecting plurality of intermediate struts 1414. The intermediate struts 1414 form a serpentine-like pattern. Points of weakness 1420 can be formed on the struts 1410 in strategic locations to facilitate controlled bending of the frame 1402. In the present aspect, these points of weakness comprise points of reduced cross-sectional area. Further, in the present aspect these points of weakness can be formed at the connection points between the rings and the longitudinal struts and the connection between the longitudinal struts and the struts of the serpentine pattern. Because of the narrow width at the connection points the longitudinal struts can flare open in the radial direction, while simultaneously causing the serpentine struts to expand radially.

When the proximal and distal rings 1404, 1406 move toward one another, such as by any of the mechanisms hereinabove described, the filter frame 1402 can assume a deployed configuration as shown in FIG. 38. The longitudinal struts can pivot radially outward, while the serpentine struts can spread apart to permit circumferential expansion.

FIG. 39 shows the filter frame 1402 covered in a filter membrane 1430. The distal end of the filter membrane can be open to permit embolic particles to enter the filter, where they can be trapped by the filter membrane.

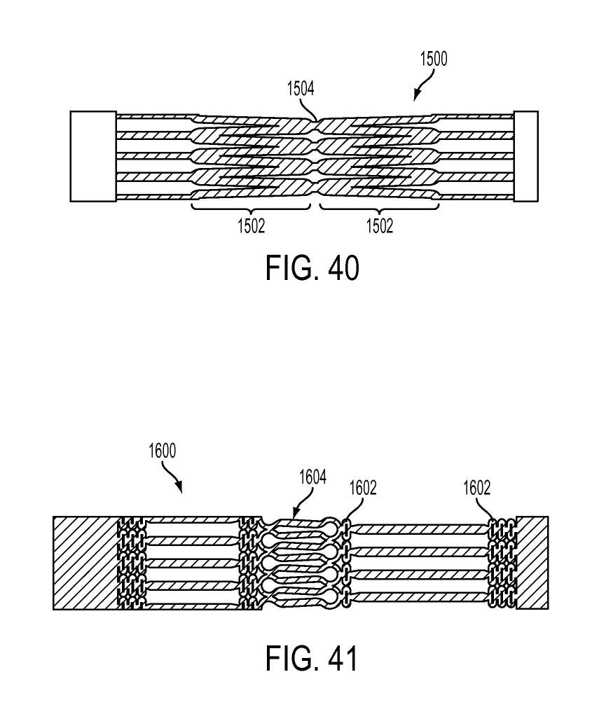

FIGS. 40 and 41 can be cylindrical projections depicting alternate frame designs. In FIG. 40, the frame 1500 can comprise two sets of intermediate struts 1502 that can form serpentine patterns. The two sets of intermediate struts 1502 can be joined by connecting members 1504. Points of weakness can be formed at strategic locations, e.g. at connections between longitudinal and intermediate struts and at the connections between the intermediate struts and the connecting members 1504.

FIG. 41 depicts another aspect of a frame 1600 in which the points of weakness can be formed by circular or oval cutouts 1602 transverse to the longitudinal axis of the struts 1604. These type of structures 1602 can provide flexibility resulting in easier opening and closing of the frame 1600. These structures 1604 can also reduce the stress induced permanent set and hence, allow the frame 1600 to retract back to its original shape. The oval and/or circular structures 1602 can also provide enough longitudinal rigidity which can urge the filter frame to open.

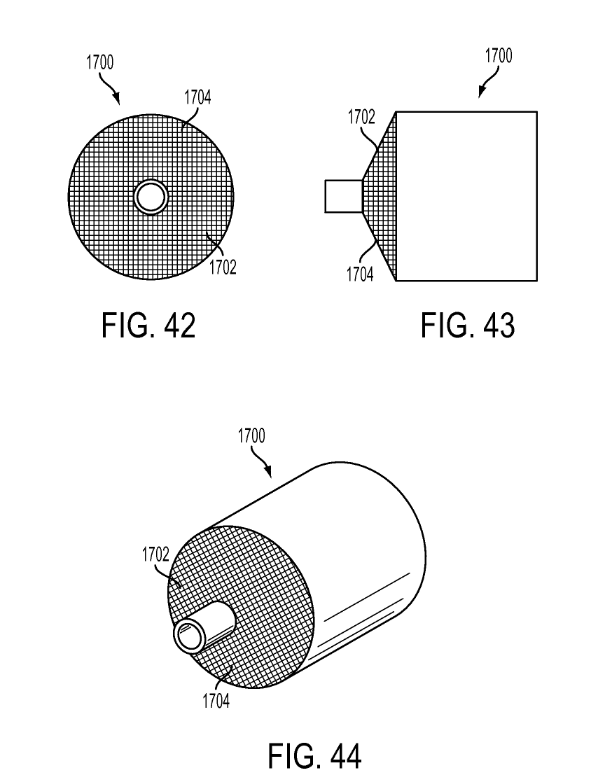

FIGS. 42-44 illustrate an aspect of a filter membrane 1700. The filter membrane 1700 can be formed in the shape of a funnel. The conical surface 1702 of the funnel can have a plurality of holes 1704 formed therein. The filter membrane 1700 can comprise semi-compliant material such as nylon or PebaxT or can comprise elastic materials such as thermoplastic elastomers or thermoset elastomers. In an alternative aspect, the filter membrane can comprise a wire mesh filter which can be formed from a wire mesh comprising a plurality of woven metallic or polymeric wires. Some examples of thermoset elastomers polyurethane and copolymers include, but are not limited to. Pellathane™, Tecothane™, Chronofles™, and the like. These materials can allow placement of the holes 1704 close to each other. In one exemplary embodiment, the size of the holes 1704 can be about 40 microns, and the holes 1704 can be placed about 40 microns apart.

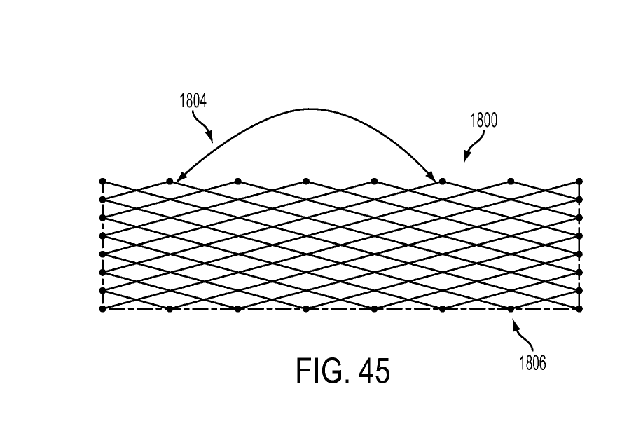

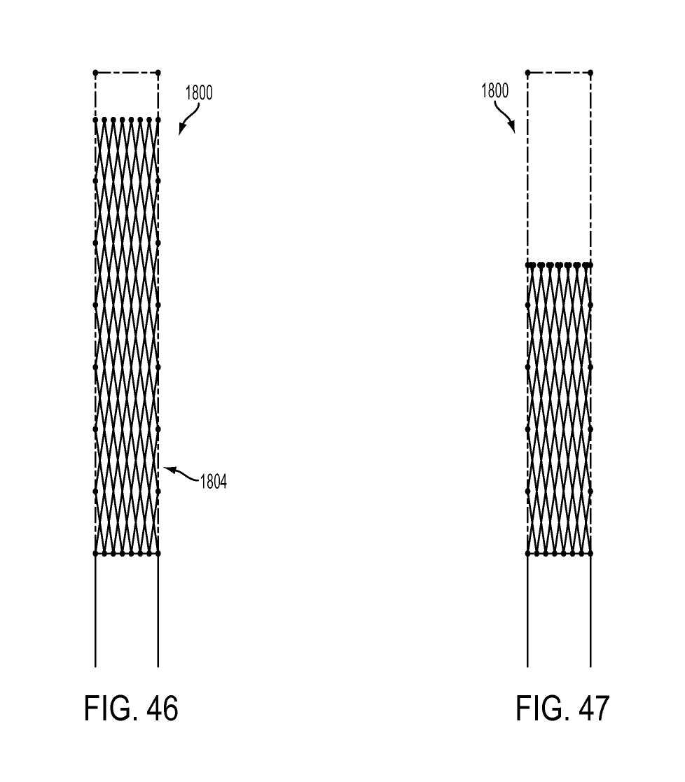

Referring now to FIGS. 44-47, yet another aspect of a frame in which the frame is formed from braided wires to form a wire mesh frame 1800 is illustrated. FIG. 44 shows a perspective view of an alternate frame design shown unrolled and flattened. In this aspect, it will be appreciated that the wire mesh frame 1800 has a generally cylindrical shape when formed. FIG. 45 shows the unformed wire mesh being stretched out over a mandrel to form a substantially cylindrical shape and FIG. 46 shows the respective ends of the formed frame design being trimmed to form a desired elongate longitudinal length. In various optional aspects, it is contemplated that the resultant trimmed cylindrically shaped frame design can be heat treated to set the shape memory in the formed position or, optionally, the trimmed frame can be compressed to a desired dimension and then heat treated to set the shape memory in the formed position (baseline closed position) or it can be expanded to a desired dimension and then heat treated to set the shape memory in the formed position (baseline open position).

In this aspect, one skilled in the art can appreciate that wire braiding techniques can be employed to form the wire mesh frame 1800 that are similar to those used to manufacture stents, closure devices, intra-vascular devices and the like. The wires can comprise, for example and without limitation, metal wires, polymer wires and the like. In one aspect, the frame can be formed from a wire braid comprising from about 12 to about 16 wires. In another aspect, the wire mesh frame un-deployed diameter 1802 can be from about 0.8 to about 1.0 mm and can be adapted to slidingly fit the catheter shaft diameter. The lead angle between the wires comprising the wire mesh from can be selected to be relatively low to allow the wire mesh frame 1800 to open to a relatively high diameter when deployed. This deployed diameter 1806 can be about 4-7 mm. The wires comprising the wire mesh frame can have a rounded profile in cross-section. The wires comprising the wire mesh frame can also be from about 0.002″ to about 0.003″ in diameter or, alternatively the wires can be flat. If the wires are selected to be flat, the wire can be further configured to be about 0.001″×0.003″ in cross-section in order to reduce the profile of the wire mesh frame.

In one embodiment, the braided wire mesh frame 1800 can be formed from 14 Nitinol or Cobalt-Chromium round wires having a 60 micron diameter and a braiding angle 1804 of about 150 degrees on a 7 mm shaft that corresponds to the maximum deployed diameter. In this aspect, the braiding angle can be defined as double (2×) the angle between the wire and the central axis. Optionally, it is contemplated that the braiding angle can be between about 1.5× and 4× or be between 1.7× and 3×. In this aspect, it is contemplated that the braided wire mesh frame 1800 can then be compressed to un-deployed diameter 1802 of about a 1 mm and heat treated to shape set the form, i.e., to set the base or unstrained shape memory in a base line closed position. Of course, it is also contemplated that the braided wire mesh frame 1800 can be heat treated to set the base or unstrained shape memory in a base line open position. It is contemplated that the wire mesh frame can form a relatively wide mesh when opened in order to allow blood flow into the filter membrane. It is also contemplated that the wire mesh frame can comprise less than 12 wires or more than 16 wires, depending on the desired inhibition or lack thereof to the flow of blood.

As shown in FIG. 16, a wire mesh frame 1800 can be incorporated into the device by joining the distal end 1808 of the wire mesh frame to a distal ring. In one aspect, the distal end of the wire mesh frame can be thermally bonded to a polymer or metallic distal ring. The distal ring can be adapted to slideably fit over the catheter shaft. The proximal end 1810 of the wire mesh frame can be attached to the proximal ring by thermal bonding as described above or other methods known to one skilled in the art. The distal and proximal rings can be of any length and diameter, but in one aspect, both the proximal and distal ends can have a length from about 0.5 to about 2.0 mm.

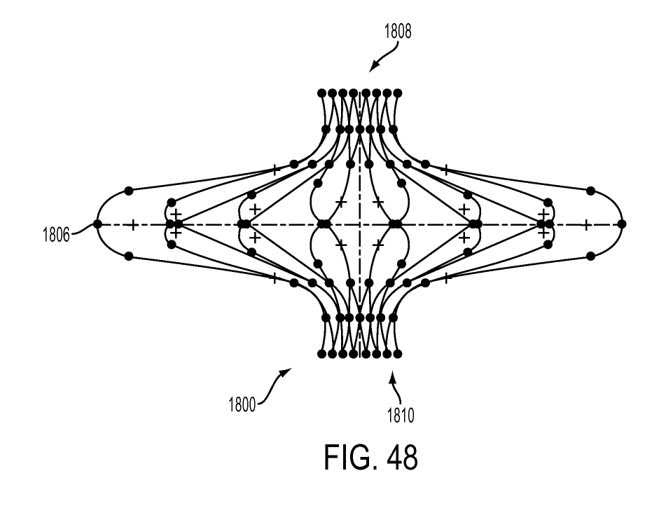

In one exemplary aspect, FIG. 47 shows an exemplary trimmed cylindrically shaped frame design that has been heat treated to set the shape memory in the baseline closed position upon application of an axial compression. For example and not meant to be limiting, the applied axial compression can be exerted by an external operator pulling on a pull wire to controllably cause the mid-portion of the trimmed cylindrically shaped frame design to expand to a desired diameter. This desired diameter is a multiple of the original diameter of the frame design in the baseline closed position.

The filter membrane 1700 and 1800 can be attached to a support frame, such as the frames 1400, 1500, or 1600 hereinabove described, such that it covers one end of the frame as well as the centrally located serpentine strut structure. The set of longitudinal struts of the filter frame can remain exposed. The filter membrane 1700 can be attached on the outside of the frame or on the inside of the frame. In addition, it is contemplated that the distal end of the membrane can be terminated at the distal ring or can extend beyond the ring to attach to the shaft of the catheter distal to the distal ring.

In the particular case of wire mesh frame 1800, the filter membrane can be attached to the wires comprising the frame 1800 at a location that is approximately equidistant between the proximal and distal ends of the wire mesh frame as it is positioned on the catheter shaft. In a further aspect, the configuration of the device depicted in FIG. 16 and described in the corresponding text can be employed with the wire mesh frame 1800. Here, a pull wire operable from outside the patient which can be attached to a distal ring of the embolic filter. When the physician exerts tension on the wire, the distal ring can be displaced proximally, bringing it closer to the proximal ring, causing the ribs to bow outward and thereby deploying the embolic mesh filter. The wire mesh frame 1800 can expand to urge the membrane edge into contact with the blood vessel wall, thus directing blood to flow through the membrane to filter any embolic particles from the blood flow. Subsequent to the procedure, the pull wire can be advanced allowing the wire mesh frame to compress back to its un-deployed state to facilitate removal of the device from the patient.

In light of the foregoing disclosure, one skilled in the art will be able to appreciate the advantages of a braided wire mesh frame 1800 relative to a laser-cut frame. The braided wire mesh frame can be more flexible, increasing the ease of navigation through tortuous anatomies to the target site. The braided wire mesh frame can also seat more tightly on the catheter shaft in an un-deployed state and lack the struts or any other projections that could potentially disengage the frame from catheter shaft during delivery or withdrawal of the device through tortuous anatomy or through previously deployed stents or other vascular devices. A braided frame can expand to create a rounded sealing edge together with the membrane edge regardless of whether it's expanded in straight or curved vessel location and creates a greater number of compression points to seal the membrane to the vessel wall around the circumference of the vessel. Further, the braided wire mesh frame more easily compresses to a smaller diameter after expansion, increasing the ease of withdrawal.

The filters herein depicted can be deployed by pulling or pushing an actuation wire or inflating an actuation balloon, depending on the type of catheter chassis being used. As the filter deploys, the serpentine struts expand circumferentially. The filter membrane is thus deployed. Upon removal of the actuation force, the filter can retract to its normally closed position.

An advantage of the filter material can be that its natural shape can be in a closed or un-deployed condition. The filter material can stretch as the filter deploys and collapse to its normal condition when the frame retracts. Therefore, the membrane has no permanent set during storage and can always be expanded to a correct size. Further, because the filter collapses under the resiliency of the filter material, the filter does not require a recovery sheath. If needed, however, a sheath may be used to further collapse the filter containing embolic debris prior to retrieval.

In an optional aspect, the filters of the disclosed aspect can be characterized by a long filter body that opposes the vessel wall over a greater area, thus reducing the chance of leakage between the filter and the vessel wall.

In each of the foregoing examples, it will be appreciated that an angioplasty balloon can be but one means for relieving a stenosis in a vessel. Stents, mechanical thrombectomy devices, or other suitable apparatus may be substituted for the angioplasty balloon and positioned on the catheter at a location proximal to the embolic filter. Thus any emboli loosened by the stent or mechanical thrombectomy device can be captured by the embolic filter in the same manner as described above with respect to the angioplasty balloon.

While the foregoing disclosed aspects comprise filter ribs of a shape memory metal such as nitinol, it can be appreciated that similar results can be obtained by using any suitable resilient material. The ribs would be formed straight, forced open by the balloon, and return to their normal shape as a result of the resiliency of the structure. Or, in the case of the aspect of FIGS. 33 and 34, the ribs would be initially formed in an open position, deformed inwardly to fit within the outer catheter, and return to their normal open position when released from the confines of the outer catheter.

Variations in the design of the filter can be also contemplated. For example, while both ends of the ribs 26 of the filter 20 can be mounted to rings 22, 24, it can be appreciated that the ends of the ribs at the fixed end of the filter can be secured directly to the catheter shaft.

Thus, implementations of the foregoing provide various desirable features. For instance, the present disclosure permits the placement of the embolic filter very close to the means for treating the stenosis. This has the effect of minimizing the “landing area” of the filter and also permits the protection of side branches, as shown in FIGS. 22-25.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An apparatus comprising:
a catheter having an elongated shaft, proximal and distal ends, and a longitudinal axis; and
a filter membrane support structure, comprising:
a first ring coaxially fixedly mounted on a distal portion of said catheter shaft;
a second ring coaxially slideably mounted on a distal portion of said catheter shaft for movement toward and away from said first ring; and
a scaffolding extending between said first and second rings, said scaffolding comprising:
first longitudinal connecting members having a first end attached to said first ring and a second end extending toward said second ring;
second longitudinal connecting members having a first end attached to said second ring and a second end extending toward said first ring;
each of said first and second longitudinal connecting members having a bifurcation formed on said second end thereof, each of said bifurcations comprising first and second branches; and
means for connecting a branch on each of said first longitudinal connecting members to a branch on an opposite one of said second longitudinal connecting members.

2. A percutaneous transluminal angioplasty device, comprising:
an elongated catheter having proximal and distal ends and an outer side wall;
an interventional device attached to the catheter adjacent the distal end thereof;
a filter attached to the elongated catheter between the interventional device and the distal end of the catheter, the filter being collapsible for insertion of the distal end of the catheter into a blood vessel, and the filter being expandable to an expanded position to capture emboli released into a bloodstream by operation of the interventional device, wherein the filter comprises:
a movable ring portion movably attached to the catheter;
a fixed ring portion immovably attached to the catheter such that the movable ring portion is movable relative to the fixed ring portion, wherein the movable ring portion is distal to the fixed ring portion;
a wire mesh frame that is formed of a shape memory material that urges the filter mesh to a ribs into a base line closed or collapsed position, a distal end of the wire mesh frame is coupled to the movable ring portion and a proximal end of the wire mesh frame is coupled to the fixed ring portion; and
a filter mesh overlying a portion of the wire mesh frame;
wherein the catheter further comprises a lumen and a port in communication with the lumen, the port comprising an aperture in the outer side wall of the catheter located distal to the fixed ring portion and proximal to the movable ring portion, and the lumen extending from a location proximate the proximal end of the catheter to the port; and
an actuator wire having proximal and distal ends, the actuator wire extending through the lumen of the catheter, and the distal end of the actuator wire exiting the lumen of the catheter through the port, the distal end of the actuator wire being attached to the movable ring portion;
wherein, when the filter is in the collapsed position, pulling on the proximal end of the wire exerts a force on the movable ring portion in the proximal direction that moves the movable ring portion toward the fixed ring portion and causes the wire mesh frame to bow outward to expand the filter to the expanded position;
wherein, when the filter is in the expanded position, releasing tension on the wire permits the shape memory of the wire mesh frame to return the wire mesh frame to the base line closed or collapsed position, collapsing the filter.

3. The percutaneous transluminal angioplasty device of claim 2, wherein the interventional device comprises an angioplasty balloon.

4. The percutaneous transluminal angioplasty device of claim 2, wherein the interventional device comprises a stent.

5. The percutaneous transluminal angioplasty device of claim 2, wherein the interventional device comprises a mechanical thrombectomy device.

6. The percutaneous transluminal angioplasty device of claim 2, wherein the shape memory material comprises Nitinol or Cobalt-Chromium.

7. The percutaneous transluminal angioplasty device of claim 2, wherein filter mesh overlies a distal portion of the wire mesh frame, and wherein, in the expanded position, the wire mesh frame bow outward, radially expanding the filter mesh.

8. The percutaneous transluminal angioplasty device of claim 2, wherein the filter mesh extends beyond the wire mesh frame in a longitudinal direction relative to the longitudinal axis of the catheter, such that a sac is formed to retain embolic particles when the filter is in the collapsed position.

9. The percutaneous transluminal angioplasty device of claim 2, wherein the wire mesh frame comprises, metal wires, polymer wires and the like.

10. The percutaneous transluminal angioplasty device of claim 9, wherein the wire mesh frame is formed from a wire braid comprising from between about 12 to about 16 wires.

11. The percutaneous transluminal angioplasty device of claim 10, wherein the wires comprising the wire mesh frame can have a rounded profile in cross-section.

12. The percutaneous transluminal angioplasty device of claim 2, wherein the wires comprising the wire mesh frame can have a flat profile in cross-section.

13. The percutaneous transluminal angioplasty device of claim 2, wherein a braiding angle between the wires of the wire mesh frame and a longitudinal axis of the wire mesh frame is a multiple between about 1.5× and 4× of the angle between the wire and the central axis when the wire is in the base line closed or collapsed position.

14. The percutaneous transluminal angioplasty device of claim 2, wherein a braiding angle between the wires of the wire mesh frame and a longitudinal axis of the wire mesh frame is a multiple between about 1.7× and 3× of the angle between the wire and the central axis when the wire is in the base line closed or collapsed position.

15. The percutaneous transluminal angioplasty device of claim 2, wherein a braiding angle between the wires of the wire mesh frame and a longitudinal axis of the wire mesh frame is a multiple of about double (2×) of the angle between the wire and the central axis when the wire is in the base line closed or collapsed position.

16. The percutaneous transluminal angioplasty device of claim 2, wherein a braiding angle between the wires of the wire mesh frame and a longitudinal axis of the wire mesh frame is a about 150 degrees.

17. The percutaneous transluminal angioplasty device of claim 2, wherein the wire mesh frame forms a relatively wide mesh when opened in order to allow blood flow into the filter membrane.

18. A percutaneous transluminal angioplasty device, comprising:
an elongated catheter having proximal and distal ends;
an interventional device attached to the catheter adjacent the distal end thereof;
a filter attached to the elongated catheter between the interventional device and the distal end of the catheter, the filter being collapsible for insertion and removal of the distal end of the catheter into a blood vessel, and the filter being expandable to an expanded position to capture emboli released into a bloodstream by operation of the interventional device, wherein the filter comprises:
a movable ring portion movably attached to the catheter;
a fixed ring portion immovably attached to the catheter such that the movable ring portion is movable relative to the fixed ring portion;
a wire mesh frame that is formed of a shape memory material that urges the filter mesh to a ribs into a base line closed or collapsed position, a distal end of the wire mesh frame is coupled to the movable ring portion and a proximal end of the wire mesh frame is coupled to the fixed ring portion; and
a filter mesh overlying a portion of the wire mesh frame;
wherein the catheter further comprises a lumen extending from a location proximate the proximal end of the catheter, to a location distal to the interventional device; and
an actuator wire having proximal and distal ends, the actuator wire extending through the lumen of the catheter, the proximal end of the actuator wire extending to a location proximate the proximal end of the catheter and the distal end of the actuator wire exiting the lumen through the side wall of the catheter at the location distal to the interventional device, the distal end of the actuator wire being attached to the movable ring portion;
wherein when the filter is in a collapsed condition, manipulating the proximal end of the wire exerts a force on the movable ring portion that moves the movable ring portion toward the fixed ring portion and causes the wire mesh frame to bow outward to the expanded position.

19. The percutaneous transluminal angioplasty device of claim 18, wherein the movable ring portion is the distal ring portion.

20. The percutaneous transluminal angioplasty device of claim 19, wherein the distal end of the actuator wire exits the lumen through the catheter side wall at a location distal to the proximal ring portion.

21. The percutaneous transluminal angioplasty device of claim 20, wherein the distal end of the actuator wire is operatively connected to the distal ring portion.

22. The percutaneous transluminal angioplasty device of claim 21, wherein pulling on the proximal end of the actuator wire draws the distal ring portion toward the fixed proximal ring portion.

23. The percutaneous transluminal angioplasty device of claim 18, wherein the interventional device comprises an angioplasty balloon.

24. The percutaneous transluminal angioplasty device of claim 18, wherein the interventional device comprises a stent.

25. The percutaneous transluminal angioplasty device of claim 18, wherein the interventional device comprises a mechanical thrombectomy device.

26. The percutaneous transluminal angioplasty device of claim 18, wherein the shape memory material comprises Nitinol or Cobalt-Chromium.

27. The percutaneous transluminal angioplasty device of claim 18, wherein filter mesh overlies a distal portion of the wire mesh frame, and wherein, in the expanded position, the ribs bow outward, radially expanding the filter mesh.

28. The percutaneous transluminal angioplasty device of claim 18, wherein the filter mesh extends beyond the wire mesh frame in a longitudinal direction relative to the longitudinal axis of the catheter, such that a sac is formed to retain embolic particles when the filter is in the collapsed position.

29. The percutaneous transluminal angioplasty device of claim 18, wherein the wire mesh frame comprises, metal wires, polymer wires and the like.

30. The percutaneous transluminal angioplasty device of claim 18, wherein the wire mesh frame is formed from a wire braid comprising from between about 12 to about 16 wires.

31. The percutaneous transluminal angioplasty device of claim 18, wherein a braiding angle between the wires of the wire mesh frame and a longitudinal axis of the wire mesh frame is a multiple between about 1.5× and 4× of the angle between the wire and the central axis when the wire is in the base line closed or collapsed position.
